(19) United States
(12) Patent Application Publication
(10) Pub. No.: US 2026/0272823 A1
(43) Pub. Date: Sep. 17, 2026

(54) INJECTABLE GEL COMPOSITION

(71) Applicant: Maman Biomedical Inc., Toronto (CA)

(72) Inventor: Latchmi Raghunanan, Toronto (CA)

(73) Assignee: Maman Biomedical Inc., Toronto (CA)

(21) Appl. No.: 19/553,662

(22) Filed: Mar. 2, 2026

Related U.S. Application Data
(60) Provisional application No. 63/764,658, filed on Feb. 28, 2025.

(51) Int. Cl. A61K 9/00; A61K 9/06; A61K 31/57; A61K 38/09; A61K 38/24; A61K 47/10; A61K 47/12; A61K 47/34; A61K 47/38

(52) U.S. Cl. CPC A61K 9/0024; A61K 9/06; A61K 31/57; A61K 38/09; A61K 38/24; A61K 47/10; A61K 47/12; A61K 47/34; A61K 47/38

(57) ABSTRACT

The present disclosure relates to an injectable gel composition for controlled, short-term delivery of fertility hormones. The injectable gel composition includes 0.5–20% w/w non-crosslinked high-molecular-weight hyaluronic acid (1000–1400 kDa) and 0.5–20% w/w methylcellulose. The high molecular weight hyaluronic acid has a molecular weight of 1000–1400 kDa. The methylcellulose has a viscosity of 100–8000 Cp. The hyaluronic acid is non-crosslinked. The hyaluronic acid and methylcellulose form a physically assembled hydrated polymer matrix through reversible physical interactions. The physically assembled hydrated polymer matrix is injectable and formed in an absence of added chemical crosslinking agents. The injectable gel composition exhibits shear-responsive flow sufficient to permit injection through a needle or microneedle lumen and recovers mechanical cohesion upon cessation of shear. The hydrated polymer matrix loses structural cohesion and disperses, dilutes, or degrades within 1–14 days following administration without forming a chemically crosslinked polymer depot.

### CROSS-REFERENCE TO RELATED APPLICATIONS

The present application claims the benefit of US Provisional Patent Application No. 63,764, 658, titled “DISSOLVABLE MICRONEEDLE PATCH FOR LARGE MOLECULE THERAPEUTIC DELIVERY” and filed on Feb. 28, 2025, all of which are hereby incorporated by reference in their entirety for all purposes.

### TECHNICAL FIELD

The present invention relates to the field of drug delivery systems and pharmaceutical formulations. More particularly, the present disclosure relates to an injectable, physically structured polymer gel composition for controlled, short-term delivery of therapeutic agents.

### BACKGROUND

Many therapeutic areas rely on large-molecule drugs for clinical efficacy. Large-molecule therapeutics include proteins, peptides, and hormone biologics. These molecules exhibit short effective lifetimes in vivo due to enzymatic degradation, renal clearance, and systemic diffusion. Rapid clearance reduces therapeutic exposure within hours. So, patients administer repeated injections to maintain efficacy. However, repeated injection creates treatment burden. Fertility and hormonal therapies require tightly regulated exposure over defined timeframes. In addition, hormone concentrations must remain within therapeutic windows. Variability in exposure disrupts treatment outcomes. Also, missed or mistimed administration compromises therapeutic success.

Formulation strategies attempt to extend drug residence at the administration site. Injectable gels and hydrogels represent one such strategy. The injectable gels include polymer solutions or suspensions that undergo physical or chemical structuring after administration. Gel formation localizes drug within a hydrated polymer matrix. The matrix attempts to slow diffusion and prolong release.

However, conventional injectable gel systems primarily regulate release through passive diffusion. Hydrated polymer networks permit rapid diffusion of large-molecule hormones. Open network structures allow early front-loaded release. In addition, increase in polymer concentration restricts diffusion but increases injection force and reduces injectability. Also, crosslink density influences injectability and diffusion simultaneously. Injectability and release behavior remain mechanistically coupled.

Some formulation approaches use long-acting depot systems to extend release duration. The depot systems rely on bulk polymer degradation, erosion, and matrix diffusion. Release becomes irreversible once administration occurs. Clinicians cannot rapidly terminate or adjust hormone exposure. Typically, the long-acting depot systems operate over weeks or months. Fertility treatment protocols require adjustable, short-cycle dosing over hours to days. Also, the depot systems exhibit burst release followed by prolonged degradation-controlled phases. Early variability arises from heterogeneous drug distribution, pore formation, hydration rate, and local tissue conditions. Further, material parameters that govern early release govern long-term degradation. Formulators cannot independently tune short-term exposure without altering total release duration. Further, the large-molecule fertility hormones require predictable, day-scale exposure rather than chronic suppression. The existing injectable gels and depot formulations do not provide controlled short-term delivery without irreversible commitment to prolonged hormone exposure.

In light of the above stated discussion, there exists a need for an injectable gel composition for enabling predictable release behavior, and avoid irreversible long-acting depot formation.

### SUMMARY

In an aspect, the present disclosure provides an injectable gel composition for controlled, short-term delivery of therapeutic agents. The injectable gel composition includes 0.5–20% w/w high molecular weight hyaluronic acid and 0.5–20% w/w methylcellulose. The high molecular weight hyaluronic acid has a molecular weight of 1000–1400 kDa. The methylcellulose has a viscosity of 100–8000 Cp. The hyaluronic acid is non-crosslinked. The hyaluronic acid and methylcellulose form a physically assembled hydrated polymer matrix through reversible physical interactions. The physically assembled hydrated polymer matrix is formed in an absence of added chemical crosslinking agents. The physically assembled hydrated polymer matrix is injectable The injectable gel composition exhibits shear-responsive flow sufficient to permit injection through a needle or microneedle lumen and recovers mechanical cohesion upon cessation of shear. The hydrated polymer matrix loses structural cohesion and disperses, dilutes, or degrades within 1–14 days following administration without forming a chemically crosslinked polymer depot.

In an embodiment of the present disclosure, the hyaluronic acid and methylcellulose are present in a weight ratio of 1:3 to 3:1.

In an embodiment of the present disclosure, the injectable gel composition further includes 0.5–10% w/w low molecular weight hyaluronic acid having a molecular weight of 20–300 kDa.

In an embodiment of the present disclosure the methylcellulose has a viscosity of 400–4000 cP.

In an embodiment of the present disclosure, the matrix loses structural cohesion within 1–7 days following administration.

In an embodiment of the present disclosure, the injectable gel composition further includes a biodegradable polymer additive configured to degrade within 1–14 days following administration.

In an embodiment of the present disclosure, the biodegradable polymer is selected from polyethylene glycol (PEG), poly(lactic-co-glycolic acid) (PLGA), polycaprolactone (PCL), or poly(lactic acid) (PLA).

In an embodiment of the present disclosure, the injectable gel composition further includes a fatty acid or fatty-acid derivative configured to modulate transient microstructural organization within the matrix.

In an embodiment of the present disclosure, the combined amount of high molecular weight hyaluronic acid and methylcellulose is 2–20% w/w of the injectable gel composition.

In an embodiment of the present disclosure, the injectable gel composition is injectable through a needle having a gauge of 18–32.

In an embodiment of the present disclosure, the hyaluronic acid and methylcellulose are combined with heated water at a temperature of 20–80°C during preparation of the injectable gel composition.

In another aspect, the present disclosure provides an injectable gel composition for controlled, short-term intradermal, subcutaneous and intramuscular drug delivery of a fertility hormone. The injectable gel composition includes 0.5–20% w/w high molecular weight hyaluronic acid and 0.5–20% w/w methylcellulose. The high molecular weight hyaluronic acid has a molecular weight of 1000–1400 kDa. The methylcellulose has a viscosity of 100–8000 Cp., the hyaluronic acid is non-crosslinked. The hyaluronic acid and methylcellulose form a physically assembled hydrated polymer matrix through reversible physical interactions. The physically assembled hydrated polymer matrix is formed in an absence of added chemical crosslinking agents. The injectable gel composition exhibits shear-responsive flow sufficient to permit injection through a needle or microneedle lumen. In addition, the injectable gel composition recovers mechanical cohesion upon cessation of shear. The fertility hormone is present in unmodified form within the hydrated polymer matrix. The hydrated polymer matrix retains the fertility hormone at an administration site. Further, the hydrated polymer matrix loses structural cohesion and disperses, dilutes, or loses structural integrity within 1–14 days following administration without formation of a covalently crosslinked polymer depot. Sustained hormone delivery is achieved over hours to days without irreversible commitment to prolonged exposure.

In an embodiment of the present disclosure, the fertility hormone is dispersed directly within the hydrated matrix.

In an embodiment of the present disclosure, at least a portion of the fertility hormone is encapsulated within biodegradable polymer particles dispersed within the matrix.

In an embodiment of the present disclosure, the biodegradable polymer is selected from polyethylene glycol (PEG), poly(lactic-co-glycolic acid) (PLGA), polycaprolactone (PCL), or poly(lactic acid) (PLA).

In an embodiment of the present disclosure, at least 75% of directly dispersed fertility hormone is released within 24 hours following administration.

In an embodiment of the present disclosure, the fertility hormone is selected from follicle stimulating hormone (FSH), luteinizing hormone (LH), gonadotropin releasing hormone (GnRH), or progesterone.

In an embodiment of the present disclosure, the injectable gel composition is configured to maintain therapeutically effective hormone levels for 1–7 days.

### BRIEF DESCRIPTION OF DRAWINGS

Having thus described the disclosure in general terms, references will now be made to the accompanying figures, wherein:

FIG. 1 illustrates an exemplary block diagram showing components of an injectable gel composition, in accordance with various embodiments of the present disclosure;

FIG. 2 illustrates a table showing representative drug-free polymer composition matrices evaluated during formulation development, in accordance with various embodiments of the present disclosure; and

FIG. 3 illustrates a table showing experimental results demonstrating influence of the injectable gel composition on resulting gel properties, in accordance with various embodiments of the present disclosure.

It should be noted that the accompanying figures are intended to present illustrations of exemplary embodiments of the present disclosure. The figures are not intended to limit the scope of the present disclosure. It should be noted that accompanying figures are not necessarily drawn to scale.

### DETAILED DESCRIPTION OF INVENTION

Some embodiments of the disclosure, illustrating all its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items. It must be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present disclosure, the preferred, systems and methods are now described. Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

While the present invention is described herein by way of example using embodiments, those skilled in the art will recognize that the invention is not limited to the embodiments described and are not intended to represent the scale of the various components. It should be understood that the detailed description thereto is not intended to limit the invention to the particular form disclosed, but on the contrary, the invention is to cover all modifications, equivalents, and alternatives falling within the scope of the present invention as defined by the appended claim. As used throughout this description, the word "may" is used in a permissive sense (i.e. meaning having the potential to), rather than the mandatory sense, (i.e. meaning must). Further, the words "a" or "an" mean "at least one” and the word “plurality” means “one or more” unless otherwise mentioned. Furthermore, the terminology and phraseology used herein is solely used for descriptive purposes and should not be construed as limiting in scope. Language such as "including," "comprising," "having," "containing," or "involving," and variations thereof, is intended to be broad and encompass the subject matter listed thereafter, equivalents, and additional subject matter not recited, and is not intended to exclude other additives, components, integers, or steps. Likewise, the term "comprising" is considered synonymous with the terms "including" or "containing" for applicable legal purposes. Any discussion of documents, acts, materials, devices, articles, and the like is included in the specification solely for the purpose of providing a context for the present invention. It is not suggested or represented that any or all of these matters form part of the prior art base or were common general knowledge in the field relevant to the present invention.

The present invention is described hereinafter by various embodiments. The invention may, however, be embodied in many different forms and should not be construed as limited to the embodiment set forth herein. Rather, the embodiment is provided so that this disclosure will be thorough and complete and will fully convey the scope of the invention to those skilled in the art. In the following detailed description, numeric values and ranges are provided for various aspects of the implementations described. The values and ranges are to be treated as examples only, and are not intended to limit the scope of the claims. In addition, a number of system architectures are identified as suitable for various facets of the implementations. The system architectures are to be treated as exemplary and are not intended to limit the scope of the invention.

All percentages, parts and ratios are based upon the total weight of the compositions and all measurements made are at about 25 ºC, unless otherwise specified.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5cells refers to groups having 1, 2, 3, 4, or 5 cells, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art. Where the context of the disclosure indicates otherwise, or is inconsistent with such an interpretation, the above-stated interpretation may be modified as would be readily apparent to a person skilled in the art. For example, in a list of numerical values such as "about 49”, “about 50”, “about 55”, means a range extending to less than half the interval(s) between the preceding and subsequent values, e.g., more than 49.5 to less than 52.5. Furthermore, the phrases "less than about" a value or "greater than about" a value should be understood in view of the definition of the term "about" provided herein.

Where a range of values is provided, it is intended that each intervening value between the upper and lower limit of that range and any other stated or intervening value in that stated range is encompassed within the disclosure. For example, if a range of 1 µm to 8 µm is stated, it is intended that 2 µm, 3 µm, 4 µm, 5 µm, 6 µm, and 7 µm are also explicitly disclosed, as well as the range of values greater than or equal to 1 µm and the range of values less than or equal to 8 µm.

The term "patient" and "subject" are interchangeable and may be taken to mean any living organism which may be treated with microneedle arrays and therapeutics of the present disclosure. As such, the terms "patient" and "subject" may include, but is not limited to, any non-human mammal, primate or human. In some embodiments, the "patient" or "subject" is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term "animal" as used herein includes, but is not limited to, humans and non-human vertebrates such as wild, domestic, and farm animals.

The term "tissue" refers to any aggregation of similarly specialized cells which are united in the performance of a particular function.

The term "disorder" is used in this disclosure to mean, and is used interchangeably with, the terms disease, condition, or illness, unless otherwise indicated.

The terms "administer," "administering" or "administration" as used herein refer to either directly administering a compound (also referred to as an agent of interest) or pharmaceutically acceptable salt of the compound (agent of interest) or a composition to a subject.

The term "treat,” “treated,” or “treating" as used herein refers to both therapeutic treatment , wherein the object is to reduce the frequency of, or delay the onset of, symptoms of a medical condition, enhance the texture, appearance, color, sensation, or hydration of the intended tissue treatment area of the tissue surface in a subject relative to a subject not receiving the compound or composition, or to otherwise obtain beneficial or desired clinical results. For the purposes of this invention, beneficial or desired clinical results include, but are not limited to, reversal, reduction, or alleviation of symptoms of a condition; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

In some embodiments, the compounds and methods disclosed herein can be utilized with or on a subject in need of such treatment, which can also be referred to as “in need thereof.” As used herein, the phrase “in need thereof” means that the subject has been identified as having a need for the particular method or treatment and that the treatment has been given to the subject for that particular purpose.

As used herein, the term “therapeutic” or “therapeutic agent” or “pharmaceutically active agent” means an agent utilized to treat, combat, ameliorate, prevent or improve an unwanted condition or disease of a patient.

The term “composition” as used herein refers to a combination or a mixture of two or more different ingredients, components, or substances.

The term “skin” as used herein refers to the thin layer of tissue forming the natural outer covering of the body of a person or animal. The skin is made of the epidermis and dermis. As used herein, the stratum corneum is the outermost layer of the epidermis and the papillary dermis is the uppermost layer of the dermis.

The term “immediate release” as used herein refers to polymers designed to release at least 75% of the therapeutically active ingredients on a timescale of about 0 hours to about 24 hours.

The term “sustained release” as used herein refers to polymers that do not release more than 75% of the therapeutically active ingredients until a time greater than about 24 hours after application.

The term “biodegradable polymer” as used herein refers to a polymer that breaks down or dissolved after introduction to the skin and release of the therapeutically active ingredient.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

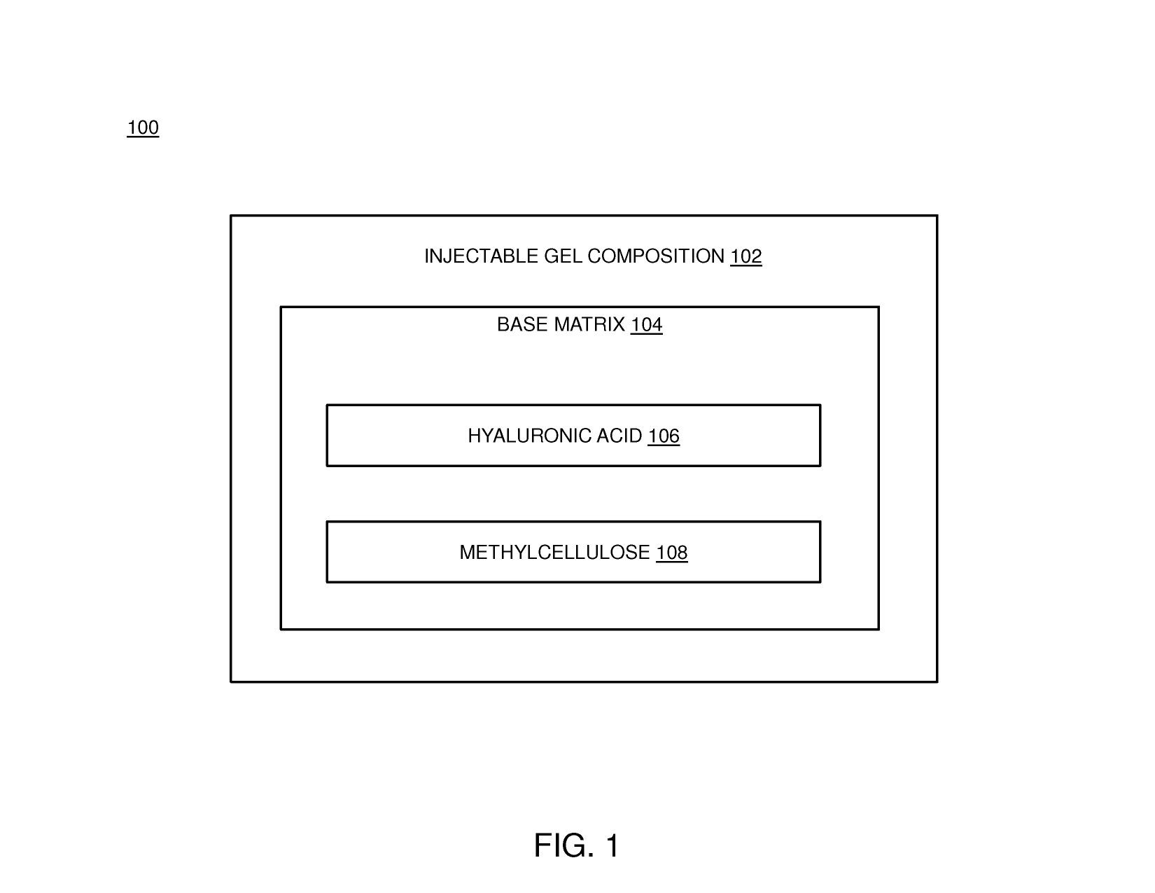

FIG. 1 illustrates an exemplary block diagram 100 showing components of an injectable gel composition 102, in accordance with various embodiments of the present disclosure. The injectable gel composition 102 provides controlled delivery of a therapeutic agent over a defined short duration. The defined short duration ranges from hours to days. The injectable gel composition 102 preserves structural integrity and bioactivity of a large-molecule drug during delivery.

The injectable gel composition 102 addresses limitations associated with conventional delivery systems. A conventional delivery system include chemically modified hormones, long-acting depots, and standard injectable gels. The conventional delivery systems fail to provide adjustable short-term exposure windows. The injectable gel composition 102 provides reversible residence and controlled dispersion aligned with clinical dosing requirements. In an embodiment, the injectable gel composition 102 is administered intradermally. In another embodiment, the injectable gel composition 102 is administered subcutaneously. In yet another embodiment, the injectable gel composition 102 is administered intramuscularly.

The injectable gel composition 102 includes a base matrix 104. The base matrix 104 forms a hydrated polymer network and supports localized drug retention. The base matrix 104 maintains structural cohesion after administration and permits dispersion after a defined interval. In addition, the base matrix 104 achieves mechanical stability without forming a permanent implant or long-acting depot.

The base matrix 104 is a physically assembled hydrated polymer network formed by hyaluronic acid 106 and methylcellulose 108. The hyaluronic acid component 106 includes a non-crosslinked hyaluronic acid polymer. The hyaluronic acid polymer has a molecular weight between 1000 kDa and 1400 kDa in certain implementations. The hyaluronic acid component 106 originates from a biologically derived or fermentation-derived source. Moreover, the hyaluronic acid component 106 remains chemically unmodified to preserve biological compatibility and structural integrity.

The hyaluronic acid 106 and the methylcellulose 108 form a physically assembled hydrated polymer matrix through reversible physical interactions. The hydrated polymer matrix corresponds to the base matrix 104. The physically assembled hydrated polymer matrix is formed in an absence of added chemical crosslinking agents. The physically assembled hydrated polymer matrix is injectable.

The hyaluronic acid component 106 exhibits physicochemical properties, and supports drug delivery performance. The hyaluronic acid component 106 is biocompatible and non-immunogenic. Further, the hyaluronic acid component 106 is highly hydrophilic and absorbs large quantities of water. The high water affinity allows the hyaluronic acid component 106 to form hydrated matrices suitable for biological environments.

The injectable gel composition 102 exhibits shear-responsive flow sufficient to permit injection through a needle or microneedle lumen and recovers mechanical cohesion upon cessation of shear. The hydrated polymer matrix loses structural cohesion and disperses, dilutes, or degrades within 1–14 days following administration without forming a chemically crosslinked polymer depot. The shear-responsive flow allows the injectable gel composition 102 to pass through fine-gauge needles or microneedle channels during administration. The hyaluronic acid component 106 supports minimally invasive delivery as the hydrated polymer network flows under applied force and stabilizes after delivery.

The water-retention capability of the hyaluronic acid component 106 promotes intimate tissue contact at an administration site. The intimate tissue contact increases localized retention of the injectable gel composition 102. The localized retention reduces premature dispersion of a therapeutic agent dispersed within the base matrix 104. Also, the hyaluronic acid component 106 undergoes rapid degradation in vivo due to enzymatic activity. A hyaluronidase enzyme cleaves polymer chains of the hyaluronic acid component 106. The enzymatic cleavage reduces molecular weight and structural cohesion.

The hyaluronic acid component 106 exhibits an effective biological half-life of approximately 12 to 24 hours in physiological environments. The short biological half-life causes rapid loss of matrix integrity. The loss of matrix integrity produces uncontrolled release of a therapeutic agent if the hyaluronic acid component 106 functions alone. The hyaluronic acid component 106 alone lacks mechanical robustness required for prolonged residence. The hyaluronic acid component 106 alone cannot maintain structural cohesion when geometry or residence duration increases. The lack of mechanical robustness limits suitability of the hyaluronic acid component 106 as a single-polymer delivery matrix. Chemically crosslinked hyaluronic acid materials provide long-term persistence but introduce limitations. The crosslinked hyaluronic acid matrix uses chemical crosslinking reactions to stabilize polymer chains. The chemical crosslinking reactions produce dense networks, resists degradation. The dense networks interfere with release control of delicate biologic drugs.

Crosslinking chemistries used in persistent hyaluronic acid materials require reactive conditions. The reactive conditions include crosslinking agents, catalysts, or non-physiological processing environments. The reactive conditions risk denaturation of protein therapeutics. Also, the reactive conditions reduce compatibility with fragile peptide hormones. The crosslinked hyaluronic acid matrix maintains structural persistence for extended durations. The extended durations exceed clinically desirable exposure windows for fertility hormone delivery. The extended persistence prevents rapid adjustment or discontinuation of therapy.

The injectable gel composition 102 avoids reliance on crosslinked hyaluronic acid architectures. The injectable gel composition 102 uses the hyaluronic acid component 106 as a structural element within a short-acting matrix system. The short-acting matrix system balances injectability, compatibility, and controlled residence.

Further, the injectable gel composition 102 uses the hyaluronic acid component 106 together with the methylcellulose component 108 to create a physically stabilized matrix (the base matrix 104). The physically stabilized matrix maintains cohesion over clinically relevant timeframes ranging from hours to days. The physically stabilized matrix disperses after the clinically relevant timeframe without forming a persistent implant.

The injectable gel composition 102 supports localized delivery routes that include intradermal administration, subcutaneous administration, and other tissue-targeted pathways. The localized delivery routes align with clinical trends favoring minimally invasive reproductive-health therapies. The localized delivery routes provide targeted exposure while limiting systemic distribution.

The injectable gel composition 102 uses physicochemical advantages of the hyaluronic acid component 106 and avoids structural limitations associated with standalone or crosslinked hyaluronic acid systems. The injectable gel composition 102 achieves a controlled short-duration delivery profile that conventional hyaluronic acid matrices fail to provide.

The hyaluronic acid component 106 provides hydration capacity and tissue compatibility. The hyaluronic acid component 106 absorbs water and forms a viscoelastic network. The viscoelastic network promotes intimate contact between the base matrix 104 and surrounding tissue. The intimate contact reduces migration of the injectable gel composition 102 from an administration site.

The methylcellulose component 108 includes a water-soluble cellulose derivative. The methylcellulose component 108 has a viscosity between 100 cP and 8000 cP. The methylcellulose component 108 functions as a rheological modifier. The methylcellulose component 108 increases viscosity and mechanical cohesion. The methylcellulose component 108 stabilizes the base matrix 104 during and after delivery. The methylcellulose component 108 exhibits non-ionic behavior. The non-ionic behavior minimizes electrostatic interaction with protein therapeutics. The minimized electrostatic interaction preserves structural stability and biological activity of a therapeutic protein dispersed within the injectable gel composition 102.

The hyaluronic acid component 106 exists in the injectable gel composition 102 at a concentration between 0.5 percent and 20 percent by weight. The methylcellulose component 108 exists in the injectable gel composition 102 at a concentration between 0.5 percent and 20 percent by weight. The injectable gel composition 102 maintains injectability within the concentration ranges. In an embodiment, the combined amount of hyaluronic acid and methylcellulose is between 2% and 20% w/w of the composition.

In certain embodiments, the hyaluronic acid component 106 and the methylcellulose component 108 maintain a weight ratio between 1:3 and 3:1. The weight ratio controls mechanical strength, extrusion force, and residence time. A balanced ratio produces a matrix that flows under shear and stabilizes after delivery.

In an embodiment, the hyaluronic acid 106 is present in an amount of 1–10% w/w. In an embodiment, the methylcellulose 108 is present in an amount of 1–10% w/w. In an embodiment, the hyaluronic acid 106 and the methylcellulose 108 are present in a weight ratio of 1:3 to 3:1. In an embodiment, the injectable gel composition 102 includes 0.5–10% w/w low molecular weight hyaluronic acid 106. The hyaluronic acid 106 has a molecular weight of 20–300 kDa.

In an embodiment, the methylcellulose 108 has a viscosity of 400–4000 cP. In an embodiment, the matrix loses structural cohesion within 1–7 days following administration

In an example, a formulation contains 2 percent hyaluronic acid and 5 percent methylcellulose, produces a cohesive gel suitable for syringe delivery. In another example, a formulation containing 1 percent hyaluronic acid and 2 percent methylcellulose produces a softer gel suitable for microneedle delivery. In a further example, a formulation containing 5 percent hyaluronic acid and 7 percent methylcellulose produces a firm gel for localized retention in subcutaneous tissue.

The hyaluronic acid component 106 provides structural hydration and localized retention. The methylcellulose component 108 provides viscosity modulation and mechanical reinforcement. The combined action of the hyaluronic acid component 106 and the methylcellulose component 108 forms the base matrix 104 through physical interactions.

The base matrix 104 represents the physically assembled hydrated polymer matrix formed through interaction between the hyaluronic acid 106 and methylcellulose 108. The base matrix 104 avoids covalent crosslinking reactions. The absence of covalent crosslinking permits reversible structural behavior and predictable dispersal.

In an embodiment, the polymers are combined with water at a temperature between 20° C and 80° C during preparation of the injectable gel composition 102.

The injectable gel composition 102 exhibits shear-thinning flow during administration. Shear-thinning flow allows the injectable gel composition 102 to pass through a needle lumen or microneedle channel. The injectable gel composition 102 recovers mechanical cohesion after shear removal. Cohesion recovery enables localized retention at a delivery site.

In an embodiment, the injectable gel composition 102 is injectable through needles having a gauge of 18–32. The injectable gel composition 102 supports short-term delivery of fertility hormones. The short-term delivery maintains therapeutic exposure without long-acting persistence. The injectable gel composition 102 disperses or dilutes within a defined timeframe after administration. The defined timeframe ranges between 1 day and 14 days in certain implementations.

The injectable gel composition 102 preserves native molecular structure of a hormone contained within the base matrix 104. The injectable gel composition 102 avoids chemical modification of the hormone. The avoidance of the chemical modification reduces regulatory complexity and preserves biological equivalence of the therapeutic agent.

The injectable gel composition 102 allows dose adjustment during treatment cycles. A clinician modifies dosage by altering injection frequency or delivered volume. The injectable gel composition 102 supports treatment flexibility as the base matrix 104 remains transient rather than persistent. The base matrix 104 achieves controlled retention through physical structuring rather than polymer degradation. The physical structuring governs drug release through short-range diffusion. The short-range diffusion produces predictable exposure over clinically relevant timeframes.

In a fertility-treatment example, a gonadotropin hormone dispersed within the base matrix 104 remains localized after injection. The localized retention maintains hormone concentration near a target tissue. Gradual dispersion of the base matrix 104 reduces hormone levels over time without abrupt discontinuation.

In another example, a peptide therapeutic incorporated within the base matrix 104 diffuses gradually as hydration increases. The gradual diffusion maintains a therapeutic concentration for several days without repeated injections.

The hyaluronic acid component 106 provides regulatory familiarity because clinical products widely use hyaluronic acid polymers. The methylcellulose component 108 provides pharmaceutical acceptability because pharmaceutical formulations widely use methylcellulose derivatives. The combined use of the hyaluronic acid component 106 and the methylcellulose component 108 reduces translational risk.

The injectable gel composition 102 establishes a structure–function relationship. In structure–function relationship, polymer identity, molecular weight, and viscosity grade determine mechanical and transport behavior. The structure–function relationship allows formulation tuning without chemical modification of a therapeutic agent.

The injectable gel composition 102 provides a transient injectable matrix. The transient injectable matrix maintains drug stability, supports localized retention, and enables adjustable delivery duration. The injectable gel composition 102 achieves desired performance characteristics. The injectable gel composition 102 conventional depot systems and standard hydrogels fail to provide.

Polymer molecular weight and methylcellulose viscosity primarily determine structural behavior of the base matrix 104 rather than total polymer concentration. Increasing total polymer concentration above an upper threshold reduces injectability of the injectable gel composition 102. The injectable gel composition 102 uses the hyaluronic acid component 106 as a primary structural material selected for established regulatory acceptance and clinical precedent. The injectable gel composition 102 intentionally addresses known limitations of the hyaluronic acid component 106 through structural design of the base matrix 104. The structural design produces a composition of matter optimized for short-term and adjustable delivery of fertility hormones. The composition of matter targets an application space not addressed by conventional hydrogels, depot systems, or chemically modified drug formulations.

The hyaluronic acid component 106 provides biological compatibility and regulatory familiarity. The physicochemical properties of the hyaluronic acid component 106 alone do not provide sufficient control for short-term delivery of large-molecule hormones. The injectable gel composition 102 includes the methylcellulose component 108 as a complementary polymer. The methylcellulose component 108 modifies rheological behavior and structural stability of the base matrix 104 without introducing permanent chemical crosslinking.

The methylcellulose component 108 includes a non-ionic and water-soluble cellulose derivative used in pharmaceutical formulations. The pharmaceutical formulations that use the methylcellulose component 108 include injectable formulations, oral suspensions, and ophthalmic preparations. The methylcellulose component 108 functions as a viscosity modifier, suspending agent, and gel-forming excipient in the pharmaceutical formulations.

The non-ionic character of the methylcellulose component 108 minimizes electrostatic interaction with protein therapeutics. The minimized electrostatic interaction reduces risk of aggregation and structural denaturation of biologic drugs dispersed within the gel composition 102. The reduced aggregation preserves biological activity of sensitive protein or peptide therapeutics.

The methylcellulose component 108 exhibits shear-thinning flow behavior in aqueous environments. The shear-thinning flow behavior allows the injectable gel composition 102 to pass through delivery devices under applied force. The methylcellulose component 108 exhibits reversible physical gelation. The reversible physical gelation allows the base matrix 104 to form a mechanically stabilized structure after administration.

In an embodiment of the present disclosure, the injectable gel composition is injectable through a needle having a gauge of 18–32. The combination of the hyaluronic acid component 106 and the methylcellulose component 108 produces a physically stabilized matrix architecture. The physically stabilized matrix architecture balances injectability, mechanical cohesion, and transient residence. The balanced properties enable controlled short-duration drug delivery without reliance on irreversible crosslinking or long-acting depot formation.

In an embodiment of the present disclosure, the methylcellulose component 108 controls post-administration cohesion of the base matrix 104 and the hyaluronic acid component 106 controls hydration and tissue interaction.

In another embodiment of the present disclosure, the methylcellulose component 108 stabilizes the base matrix 104 during injection and the hyaluronic acid component 106 promotes localized retention after delivery.

The injectable gel composition 102 uses complementary polymer functions to achieve controlled performance. The complementary polymer functions produce a delivery platform capable of adjustable exposure, localized retention, and predictable dissipation. The injectable gel composition 102 achieves the properties without chemical modification of a therapeutic agent and without formation of a persistent implant.

The methylcellulose component 108 increases structural cohesion of the base matrix 104 through physical polymer interactions. The physical polymer interactions include chain entanglement and thermo responsive structuring within an aqueous environment. The physical polymer interactions increase residence time of the injectable gel composition 102 after administration without requiring chemical crosslinking.

The methylcellulose component 108 stabilizes the base matrix 104 through reversible structuring rather than irreversible chemical bonding. The distinction is important for delivery of fertility hormones because fragile protein therapeutics exhibit sensitivity to reactive chemistries and non-physiological processing conditions.

Chemical crosslinking approaches used in long-acting depots employ reactive agents and processing environments. The reactive agents and processing environments can denature proteins or alter molecular conformation. The injectable gel composition 102 avoids the conditions as the base matrix 104 forms through physical interactions instead of chemical reactions.

The methylcellulose component 108 forms hydrated gels, and remains permeable to diffusion. The diffusion-permissive structure allows therapeutic agents to migrate through the hydrated network without long-term entrapment. The diffusion-permissive behavior makes the methylcellulose component 108 suitable for applications requiring transient stabilization rather than prolonged release. The transient stabilization supports delivery durations ranging from hours to days. The transient stabilization differs from conventional depot technologies and maintains release for weeks or months.

The methylcellulose component 108 modulates viscosity, injectability, and short-term residence of the injectable gel composition 102. The modulation occurs through adjustment of polymer concentration, viscosity grade, or molecular architecture. The adjustable parameters allow tuning of mechanical and transport behavior without creating irreversible delivery characteristics.

The selection of the methylcellulose component 108 represents a deliberate material design choice. The material design introduces temporary physical stabilization within the hydrated base matrix 104. The temporary physical stabilization provides mechanical support and tunable rheology and preserves biological activity of a therapeutic agent.

In an embodiment of the present disclosure, the methylcellulose component 108 increases cohesion of the base matrix 104 during the first twenty-four hours after administration. In another embodiment of the present disclosure, the methylcellulose component 108 maintains injectability of the injectable gel composition 102 and extends residence time of the base matrix 104 within a localized tissue region.

The injectable gel composition 102 uses the methylcellulose component 108 as a transient structural regulator that enables controlled short-duration delivery. The injectable gel composition 102 achieves adjustable exposure profiles compatible with fertility treatment cycles without forming persistent depots or requiring chemical modification of a therapeutic agent.

In an embodiment, the injectable gel composition 102 includes 0.5 to 20% w/w HMW hyaluronic acid, 1-10% w/w HMW hyaluronic acid or 1-5% w/w HMW hyaluronic acid.

In another embodiment, the injectable gel composition 102 includes 1% w/w HMW hyaluronic acid.

In yet another embodiment, the injectable gel composition 102 includes 2% w/w HMW hyaluronic acid.

In yet another embodiment, the injectable gel composition 102 includes 3%, 4%, 5%, 6% 7%, 8%, 9% or 10% w/w HMW hyaluronic acid.

In yet another embodiment, the injectable gel composition 102 includes low molecular weight (LMW) hyaluronic acid, optionally 0.5 to 20% w/w LMW hyaluronic acid or 1-10% w/w LMW hyaluronic acid.

In yet another embodiment, the injectable gel composition 102 includes 1%, 2%, 3%, 4%, 5%, 6%, 7%, 8%, 9% or 10% w/w LMW hyaluronic acid.

In an embodiment, the hyaluronic acid (HMW and/or LMW) hyaluronic acid is non-crosslinked hyaluronic acid.

In an embodiment, the methylcellulose includes methylcellulose with the viscosity between 100 cP and 8000 cP. In some embodiments, the injectable gel composition 102 includes 0.5 to 20% w/w methylcellulose or 1-10% w/w methylcellulose.

In an embodiment, the injectable gel composition 102 can include 1%, 2%, 3%, 4%, 5%, 6% , 7%, 8%, 9% or 10% w/w methylcellulose.

In an embodiment, the methylcellulose 108 has a viscosity of between 100 cP and 10,000 cP.

In an embodiment, the methylcellulose has a viscosity of 400 cP.

In some embodiment, the methylcellulose has a viscosity of 4000 cP.

The injectable gel composition 102 exhibits temperature-responsive and shear-responsive phase behavior and supports controlled delivery performance. The base matrix 104 transitions between a flowable state and a cohesive state depending on mechanical and thermal conditions. The phase behavior allows administration through narrow delivery devices with maintaining localized retention after placement.

The injectable gel composition 102 exists in a low-viscosity state before administration. The low-viscosity state permits extrusion through a fine-gauge syringe or microneedle channel under applied force. The low-viscosity state results from alignment and temporary disentanglement of polymer chains within the base matrix 104 during shear.

The injectable gel composition 102 undergoes a reversible structural transition after delivery. The reversible structural transition occurs when applied shear forces cease. The polymer chains within the base matrix 104 re-establish physical associations and entanglement. The re-established associations increase cohesion and mechanical stability of the injectable gel composition 102 at an administration site.

The phase transition of the injectable gel composition 102 occurs through physical interactions that include chain entanglement, hydration-driven structuring, and polymer network reorganization. The physical interactions allow repeated transitions between flowable and cohesive states without chemical modification.

The temperature of a physiological environment promotes stabilization of the base matrix 104 after delivery. A body temperature environment increases hydration and intermolecular association of polymer chains. The increased hydration enhances structural cohesion of the injectable gel composition 102 without creating a permanent implant.

The injectable gel composition 102 remains fluid or semi-fluid at room temperature in certain embodiments. The injectable gel composition 102 increases cohesion at physiological temperature after administration. The temperature-dependent increase in cohesion improves localized retention of the base matrix 104 within tissue.

In an embodiment of the present disclosure, the injectable gel composition 102 exhibits a phase transition temperature within a physiological range that supports stabilization after administration. In another embodiment of the present disclosure, the injectable gel composition 102 maintains injectability at room temperature and forms a mechanically stable matrix at body temperature.

The mechanical strength of the base matrix 104 depends on formulation parameters. The formulation parameters include polymer concentration, molecular weight, and viscosity grade. Adjustment of the formulation parameters controls residence duration and structural firmness of the injectable gel composition 102.

In an embodiment, a formulation containing a higher viscosity methylcellulose component 108 produces a stronger cohesive matrix after injection.

In another embodiment, a formulation containing a lower polymer concentration produces a softer matrix. The softer matrix disperses more rapidly after administration.

In yet another embodiment, a formulation containing a higher proportion of the hyaluronic acid component 106 increases hydration capacity and prolongs localized residence.

The reversible transition between flowable and cohesive states enables localized drug retention without long-term depot formation. The reversible transition allows the injectable gel composition 102 to remain transient and still maintaining short-term structural stability. The transient stability supports delivery durations aligned with clinical treatment windows.

The injectable gel composition 102 uses reversible phase behavior to control retention and release characteristics. The reversible phase behavior enables shaping of drug exposure over hours to days. The injectable gel composition 102 achieves localized delivery and avoids irreversible commitment to prolonged exposure.

In an embodiment, the injectable gel composition 102 may include one or more additives. The one or more additives modify performance of the base matrix 104. In addition, the one or more additives refine mechanical behavior, drug retention, and short-term release characteristics of the injectable gel composition 102. Further, the one or more additives function as secondary components.

In an embodiment, the injectable gel composition 102 may include one or more biodegradable polymer additives configured to degrade within 1–14 days following administration. In an embodiment, the biodegradable polymer is selected from polyethylene glycol (PEG), poly(lactic-co-glycolic acid) (PLGA), polycaprolactone (PCL), or poly(lactic acid) (PLA).

In an embodiment, the injectable gel composition 102 includes a fatty acid or fatty-acid derivative configured to modulate transient microstructural organization within the matrix.

In an embodiment, the one or more additives include biodegradable polymers selected to support short-duration functional roles. The biodegradable polymers include polyethylene glycol, polycaprolactone, polylactic acid, and polylactic-co-glycolic acid. The biodegradable polymers possess molecular weights and architectures selected to degrade or lose structural integrity within hours to days.

The one or more additives may degrade through hydrolysis, enzymatic activity, or physical reorganization after administration. The degradation time scale aligns with therapeutic windows required for fertility or hormonal treatments. The degradation behavior prevents formation of long-acting depots or persistent implants.

In an embodiment of the present disclosure, a biodegradable polymer additive increases mechanical cohesion of the base matrix 104 during an early post-administration period.

In another embodiment of the present disclosure, a biodegradable polymer additive modulates short-range diffusion of a therapeutic agent within the injectable gel composition 102.

In another embodiment, the one or more additives may include lipid-based materials. The lipid-based materials include fatty acids, fatty alcohols, esters, phospholipids, or amphiphilic lipid compounds. The lipid-based materials modify local microstructure within the base matrix 104. The modified microstructure alters drug partitioning behavior and transient hydrophobic association. In addition, the lipid-based materials provide reversible stabilization of protein or peptide therapeutics dispersed within the injectable gel composition 102. The reversible stabilization delays diffusion of a therapeutic agent without imposing long-term retention. The delayed diffusion smooths early exposure profiles after administration.

In an embodiment of the present disclosure, the fatty acid additive forms transient microdomains within the base matrix 104. The transient microdomains temporarily associate with a therapeutic protein and slow initial diffusion.

In another embodiment of the present disclosure, the lipid additive enhances structural uniformity of the base matrix 104 and improves dose distribution. The one or more additives may form encapsulation structures around the therapeutic agent. The encapsulation structures protect labile protein therapeutics before and after administration. The encapsulation structures reduce uncontrolled early diffusion and provide staged release behavior. Further, the encapsulation structures degrade or dissociate within short time intervals. The short time intervals correspond to hours or days rather than weeks or months. The short degradation interval preserves clinical ability to adjust or discontinue therapy rapidly.

The one or more additives operate as modulatory elements rather than bulk structural elements. The one or more additives provide localized and tunable influence over mechanical response, transport behavior, and microenvironmental interactions.

The injectable gel composition 102 maintains injectability and biocompatibility even when the one or more additives are present. The one or more additives remain compatible with large-molecule fertility hormones and do not interfere with biological activity. Further, the one or more additives expand formulation flexibility without altering core functional behavior of the base matrix 104.

The injectable gel composition 102 selects the one or more additives based on degradation or dissociation profiles consistent with short-duration delivery. The selection of the one or more additives ensures the injectable gel composition 102 remains fundamentally distinct from long-acting depot systems. The injectable gel composition 102 addresses clinical requirements for adjustable, short-term hormone exposure, maintaining structural tunability and localized delivery.

The injectable gel composition 102 incorporates one or more therapeutic agents within the base matrix 104. The therapeutic agents include but may not be limited to fertility hormones, endocrine regulators, peptide therapeutics, or protein biologics. The therapeutic agents possess molecular weights characteristic of large-molecule drugs. The large molecular weight requires stabilization within a delivery matrix to maintain biological activity.

In an embodiment, the combined amount of high molecular weight hyaluronic acid 106 and the methylcellulose 108 is 2–20% w/w of the injectable gel composition.

In an embodiment, the fertility hormone is selected from follicle stimulating hormone (FSH), luteinizing hormone (LH), gonadotropin releasing hormone (GnRH), or progesterone.

In an embodiment, the fertility hormone is dispersed directly within the hydrated matrix.

In an embodiment, at least a portion of the fertility hormone is encapsulated within the biodegradable polymer particles dispersed within the base matrix 104. In an embodiment, the biodegradable polymer is selected from polyethylene glycol (PEG), poly(lactic-co-glycolic acid) (PLGA), polycaprolactone (PCL), or poly(lactic acid) (PLA).

The injectable gel composition 102 accommodates the therapeutic agents. In an embodiment, the therapeutic agents include follicle-stimulating hormone, luteinizing hormone, gonadotropin-releasing hormone analogues, progesterone, or related endocrine agents. The base matrix 104 supports incorporation of the therapeutic agents without chemical modification. The absence of chemical modification preserves native molecular structure and pharmacological function.

The injectable gel composition 102 maintains stability of the therapeutic agents during storage and after administration. The hydrated environment of the base matrix 104 protects protein conformation and reduces aggregation. The non-ionic character of the methylcellulose component 108 minimizes electrostatic interactions that could destabilize sensitive biologics.

The injectable gel composition 102 distributes the therapeutic agents uniformly throughout the base matrix 104. The uniform distribution produces consistent local concentration and predictable diffusion behavior. In addition, the uniform distribution reduces localized overdosing or underexposure within tissue.

In an embodiment of the present disclosure, the therapeutic agents disperse directly within the hydrated network of the base matrix 104. In another embodiment of the present disclosure, the therapeutic agents associate with dispersed additives, and temporarily retain the therapeutic agents through reversible physical interactions. In an embodiment, the additives may include biodegradable polymers, and assist drug incorporation. In an embodiment, the biodegradable polymers include polyethylene glycol, polycaprolactone, polylactic acid, polylactic-co-glycolic acid, or related derivatives. The biodegradable polymers degrade or dissolve over short timeframes compatible with transient delivery.

The biodegradable polymers form dispersed phases, particulate domains, or microstructures within the base matrix 104. The dispersed phases temporarily couple the therapeutic agents to structural elements of the injectable gel composition 102. The temporary coupling modulates diffusion rate without creating a persistent depot.

In an embodiment of the present disclosure, a biodegradable polymer additive forms microscale domain, and holds a portion of the therapeutic agents. The microscale domains release the therapeutic agents as the biodegradable polymer dissolves.

In another embodiment of the present disclosure, a dispersed polymer particle slows initial diffusion of the therapeutic agents and produces a smoother exposure profile. In an embodiment, the additives may include lipid-based materials. The lipid-based materials influence drug stability. In an embodiment, the lipid-based materials include fatty acids, phospholipids, lipid esters, or amphiphilic lipid compounds. The lipid-based materials form transient compartments within the base matrix 104.

The transient compartments stabilize sensitive therapeutic agents by providing localized microenvironments. The localized microenvironments reduce denaturation and aggregation of protein drugs. The transient compartments disassemble under physiological conditions without permanent residue.

In an embodiment of the present disclosure, a lipid additive forms an emulsified domain. The emulsified domain encapsulates a fraction of the therapeutic agents. The encapsulated fraction releases gradually as the emulsified domain destabilizes. In another embodiment of the present disclosure, the fatty acid additive associates reversibly with a peptide therapeutic and delays early diffusion.

The injectable gel composition 102 selects the one or more additives based on compatibility with short-duration delivery. The one or more additives degrade, reorganize, or dissipate within hours to days. The short functional lifetime prevents formation of long-acting depots and maintains clinical flexibility.

The injectable gel composition 102 provides a platform for incorporating the therapeutic agents and maintain stability, homogeneity, and predictable release behavior. The injectable gel composition 102 achieves the controlled short-term delivery without requiring irreversible structural matrices or the chemical modification of active pharmaceutical ingredients.

The injectable gel composition is used for controlled, short-termintradermal, subcutaneous and intramuscular drug delivery of a fertility hormone to a subject. The injectable gel composition includes a 0.5–20% w/w high molecular weight hyaluronic acid 106 having the molecular weight of 1000–1400 kDa and 0.5–20% w/w methylcellulose 108 having the viscosity of 100–8000 cP. The hyaluronic acid 106 is non-crosslinked. The injectable gel composition 102 forms the physically assembled hydrated polymer matrix. The fertility hormone is present in unmodified form within the hydrated polymer matrix. The hydrated polymer matrix retains the fertility hormone at the administration site. The hydrated polymer matrix disperses, dilutes, or loses structural integrity within 1–14 days following administration. A sustained hormone delivery is achieved over hours to days without irreversible commitment to prolonged exposure.

The injectable gel composition 102 exhibits shear-responsive flow sufficient to permit injection through a needle or microneedle lumen. In addition, the injectable gel composition 102 recovers mechanical cohesion upon cessation of shear. The fertility hormone is present in unmodified form within the hydrated polymer matrix. The hydrated polymer matrix retains the fertility hormone at an administration site. Further, the hydrated polymer matrix loses structural cohesion and disperses, dilutes, or loses structural integrity within 1–14 days following administration without formation of a covalently crosslinked polymer depot.

In an embodiment, at least 75% of directly dispersed fertility hormone is released within 24 hours following administration. In an embodiment, the injectable gel composition 102 is configured to maintain therapeutically effective hormone levels for 1–7 days. In an embodiment, the one or more additives may encapsulate the one or more therapeutic agents before or during formation of the injectable gel composition 102. The encapsulation provides structural protection and physical separation of drug populations within the base matrix 104. In addition, the encapsulation stabilizes labile therapeutics during handling, storage, and administration.

The encapsulation uses biodegradable materials selected for short functional lifetimes. The biodegradable materials degrade, dissolve, or disassemble within hours to days under physiological conditions. The short-lived behavior prevents formation of persistent depots or irreversible delivery architectures.

The injectable gel composition 102 encapsulates different therapeutic agents using distinct encapsulation materials or structures. The distinct encapsulation structures allow independent control of release profiles within a single formulation. The independent control supports flexible formulation design and preserves native chemical identity of each therapeutic agent.

In an embodiment of the present disclosure, a first fraction of the therapeutic agent remains freely dispersed within the base matrix 104, and a second fraction resides within encapsulation structures. The freely dispersed fraction releases rapidly, and the encapsulated fraction releases gradually. In another embodiment of the present disclosure, two different therapeutic agents occupy separate encapsulation structures that degrade at different rates.

In an embodiment, the injectable gel composition 102 may include pharmaceutically acceptable excipients. The pharmaceutically acceptable excipients include aqueous carriers, buffering agents, salts, tonicity modifiers, or stabilizing compounds suitable for injectable formulations. Further, the pharmaceutically acceptable excipients support formulation stability, maintain osmotic balance, and preserve compatibility with sensitive biologic drugs. The pharmaceutically acceptable excipients function as supportive components, and maintain physicochemical stability without imposing long-term structural persistence.

The one or more additives perform multiple formulation-support functions. The one or more additives improve injectability and formulation robustness of the injectable gel composition 102. In addition, the one or more additives enhance short-term mechanical cohesion of the base matrix 104 without permanent crosslinking. Further, the one or more additives stabilize protein or peptide therapeutics against aggregation or denaturation. Also, the one or more additives enable structural compartmentalization within the base matrix 104. The one or more additives provide transient formulation-level control consistent with short-duration therapeutic applications. Furthermore, the injectable gel composition 102 uses the one or more additives as temporary modulators that refine performance without altering core structural behavior. The injectable gel composition 102 maintains short-term delivery characteristics and allows tuning of stability, release profile, and mechanical properties through selection of additive type and concentration.

In an embodiment, the injectable gel composition 102 is configured for administration via a microneedle device or microneedle patch. The shear-thinning and physically assembled nature of the gel permits flow through microneedle bores, microchannels, or dissolvable microneedle matrices under applied force, and allows recovery of mechanical cohesion following delivery into tissue.

The injectable gel composition 102 delivery through the microneedle patch enables localized subcutaneous, intradermal or transdermal placement of the gel without premature solidification, clogging, or loss of structural integrity. The microneedle-mediated administration may serve as an alternative to conventional hypodermic injection for transferring the injectable gel composition into a subject. At the same time, the microneedle-mediated administration preserves controlled short-term residence, localized retention, and bioactivity of incorporated therapeutic agents.

In certain embodiments, the injectable gel composition 102 may be utilized as a matrix material for fabrication of dissolvable microneedle patches. The hydrated polymer matrix formed by the physical interaction between the high molecular weight hyaluronic acid 106 and the methylcellulose 108 may be cast into microneedle molds and subsequently dried or otherwise processed to generate solidified microneedle arrays. In such embodiments, the shear-thinning and physically assembled nature of the gel facilitates uniform mold filling, reduced air entrapment, and consistent structural formation during casting. Upon drying or lyophilization, the polymer matrix transitions from a hydrated gel state to a solidified form and at the same time, retain non-covalent polymer architecture. The resulting microneedle structures remain fully dissolvable upon exposure to physiological fluid following insertion into skin. The therapeutic agents are incorporated into the gel prior to casting.

The formed microneedles includes a solidified matrix. The solidified matrix contains dispersed and/or encapsulated drug fractions. Upon administration, the microneedles dissolve within tissue and release the therapeutic agent in accordance with the staged release behavior described herein. After dissolution of the microneedle structures, external portions of the supporting substrate may be removed without leaving rigid delivery components within tissue.

The use of the injectable gel composition 102 as a precursor matrix for the dissolvable microneedles represents an optional manufacturing embodiment and does not alter the fundamental structure of the hydrated polymer matrix.

In an embodiment, the polymers are combined to form a physically stabilized gel network through non-covalent interactions, polymer chain entanglement, and hydration-driven structuring. The network is formed through reversible physical associations, permits structural cohesion under physiological conditions and maintains flowability under applied shear.

In an embodiment, the hyaluronic acid 106 contributes toward tissue compatibility, hydration capacity, and favorable biological interaction at an administration site. The properties support localized residence of the injectable gel composition 102 and reduce immunogenic response following administration. The hydrophilic character of the hyaluronic acid 106 promotes water uptake and formation of a hydrated microenvironment suitable for maintaining structural integrity of sensitive therapeutic agents.

In an embodiment, the methylcellulose 108 contributes reversible viscosity modulation and mechanical cohesion. The methylcellulose component 108 stabilizes the hydrated matrix for a defined duration following administration and allows the injectable gel composition 102 to remain injectable when subjected to shear stress. The reversible rheological behavior permits extrusion through delivery devices and enables the post-administration structural recovery.

In an embodiment, the resulting polymer matrix is configured to degrade, disperse, or dissipate under physiological conditions within a time period ranging from hours to days. The transient persistence aligns with therapeutic regimens requiring adjustable or interruptible exposure, such as fertility treatment cycles involving short-term hormone administration.

In an embodiment, the composition provides functional decoupling of injectability, local residence, and drug-release behavior. In contrast to conventional depot systems and solid hydrogels in which these parameters are mechanistically linked, the present composition permits independent modulation of these properties. Injectability is governed primarily by shear-dependent rheological behavior during administration. The post-administration residence and release characteristics are governed primarily by hydration dynamics and short-term physical stabilization of the matrix.

In an embodiment, the decoupled structure–function relationship enables predictable short-duration exposure of a therapeutic agent without irreversible commitment to prolonged delivery. The behavior permits clinical adjustment of dose or exposure duration through modification of administration frequency or volume.

In an embodiment, the injectable gel composition 102 is compatible with subcutaneous, intradermal, or localized tissue administration routes. The injectable gel composition 102 may be delivered using syringe-based delivery systems, cannulas, microneedle-based devices, or combinations of the above. The compatibility with multiple delivery modalities allows use of a single formulation across different administration platforms without requiring device-specific reformulation.

In certain embodiments, the injectable gel composition 102 avoids chemical modification of an active pharmaceutical ingredient and avoids long-acting depot architectures. The preservation of the native molecular form of the therapeutic agent maintains biological activity and reduces formulation-induced structural alteration. In addition, avoidance of the persistent depot structures improves clinical flexibility, supports adjustable dosing regimens, and aligns with safety and therapeutic requirements associated with controlled ovarian stimulation and other hormone-regulated treatment protocols.

In certain embodiments, the injectable gel composition 102 is prepared by hydrating the hyaluronic acid 106 and the methylcellulose 108 under controlled thermal conditions to generate a physically assembled polymer matrix. The high molecular weight hyaluronic acid 106 (1000–1400 kDa) is dissolved in approximately ten times its equivalent volume of water at a temperature of about 65°C. The solution is allowed to cool to room temperature to permit hydration and formation of a viscoelastic polymer network. In certain embodiments, the methylcellulose 108 having the viscosity grade between 100–8000 cP is dispersed in approximately ten times its equivalent volume of water at a temperature of about 65°C. The dispersion is gently agitated during cooling to room temperature to ensure uniform hydration and dissolution.

In certain embodiments, mixed polymer gels are prepared by first combining dry hyaluronic acid and methylcellulose powders in pre-determined weight ratios, followed by addition of approximately ten times the equivalent volume of water at about 65°C. The mixture is cooled to room temperature with gentle agitation to promote uniform hydration and polymer chain entanglement. The resulting composition forms a hydrated matrix through non-covalent polymer interactions, such as chain entanglement and hydration-driven structuring, without chemical crosslinking or covalent modification.

In various embodiments, the hyaluronic acid 106 is present at 0.5–20% w/w and the methylcellulose 108 is present at 0.5–20% w/w. The combined polymer content is 2–20% w/w. The hyaluronic acid 106 and the methylcellulose 108 weight ratio ranges from 1:3 to 3:1. The formulations within the ranges produce cohesive hydrated matrices exhibiting shear-responsive flow and post-shear structural recovery. In certain embodiments, low molecular weight hyaluronic acid (20–300 kDa) is incorporated at 0.5–20% w/w to modulate rheological and flow properties of the hydrated matrix. In an embodiment, active pharmaceutical ingredients may be incorporated into the hydrated matrix using one or more of the following approaches namely, direct dispersion and encapsulation prior to incorporation.

In the direct dispersion approach, the therapeutic agent is directly stirred into the pre-formed hydrated polymer matrix under controlled mixing conditions to achieve uniform dispersion.

In the encapsulation approach, the therapeutic agent is first encapsulated within biodegradable polymer particles. The encapsulated material is then gently mixed into the hydrated matrix to generate a composition comprising both dispersed and encapsulated fractions.

In a mixed approach, a first fraction of the therapeutic agent is directly dispersed within the matrix, and a second fraction is introduced in encapsulated form. The approaches enable modulation of early diffusion and sustained release behavior without forming a persistent depot.

The gel performance is evaluated based on injectability, gel–sol time, firmness, and mechanical response. The injectability is assessed using a 1 mL syringe fitted with a 30-gauge needle. Flow behavior under manual thumb pressure is evaluated to determine extrusion feasibility. Formulations exhibiting shear-responsive flow permit passage through fine-gauge needles and recover cohesion upon cessation of shear.

In certain embodiments, the injectable gel composition 102 is injectable through needles having the gauge between 18 and 32. Gel–sol time is measured as the time required for a 10 mL sample of gel to become flowable after inversion in a container of fixed geometry. The gel-sol time parameter reflects structural cohesion under static conditions.

Gel hardness is measured as the incremental weight required to flatten a 0.25 g gel sphere into a 1 mm thick sheet. The gel hardness measurement reflects resistance to compressive deformation.

Firmness is evaluated by forming a 0.25 g sphere of gel and observing its ability to retain shape:

5 = retains spherical form

4 = deforms under slight stress

3 = loses structural peak within 30 seconds

2 = threads with visible peak

1 = liquid

The metrics collectively characterize the structure–function relationship of the hydrated polymer network.

Comparative evaluation of polymer concentrations demonstrates that the hyaluronic acid 106 alone does not consistently yield matrices exhibiting prolonged cohesion and acceptable injectability. The methylcellulose 108 alone does not consistently provide balanced gel firmness and shear responsiveness. Combination of the hyaluronic acid 106 and the methylcellulose 108 yields gel properties consistent with cooperative physical interaction between polymers. Addition of low molecular weight hyaluronic acid 106 modifies the rheological behavior in a manner that may improve the injectability despite increased total solids content. The data supports that the gel behavior depends on relative polymer ratio, molecular weight, and viscosity grade rather than total polymer concentration alone.

For drug-loaded gel matrices prepared as described above, the release characteristics may be evaluated by incubating defined quantities of gel in aqueous media at approximately 37° C. At pre-determined time intervals, aliquots of surrounding media are withdrawn and analyzed by UV-visible spectroscopy to quantify drug concentration. Withdrawn volumes are replaced with fresh medium, and sampling is continued until matrix dissolution is complete or drug concentration falls below detection limits.

In certain embodiments, directly dispersed fractions exhibit early release, and the encapsulated fractions exhibit extended release consistent with degradation or dissolution of biodegradable carrier material. Matrix dispersion or loss of structural integrity occurs within approximately 1–14 days under physiological conditions.

Further, in vitro biocompatibility evaluation is done where representative formulations may be evaluated using relevant cell cultures. Cells are exposed to gel formulations under controlled conditions. Cell viability and functional biological response may be measured at multiple time points to assess compatibility and biological performance of the formulation.

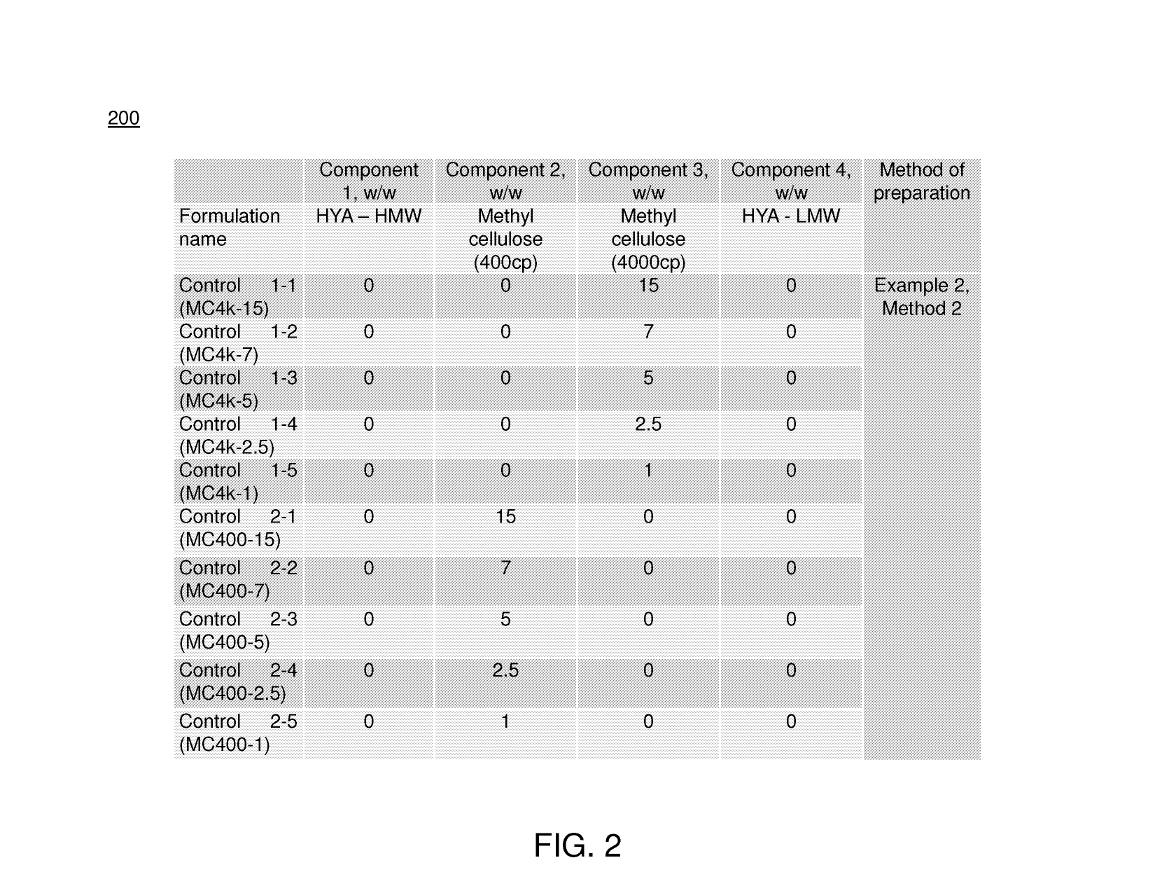

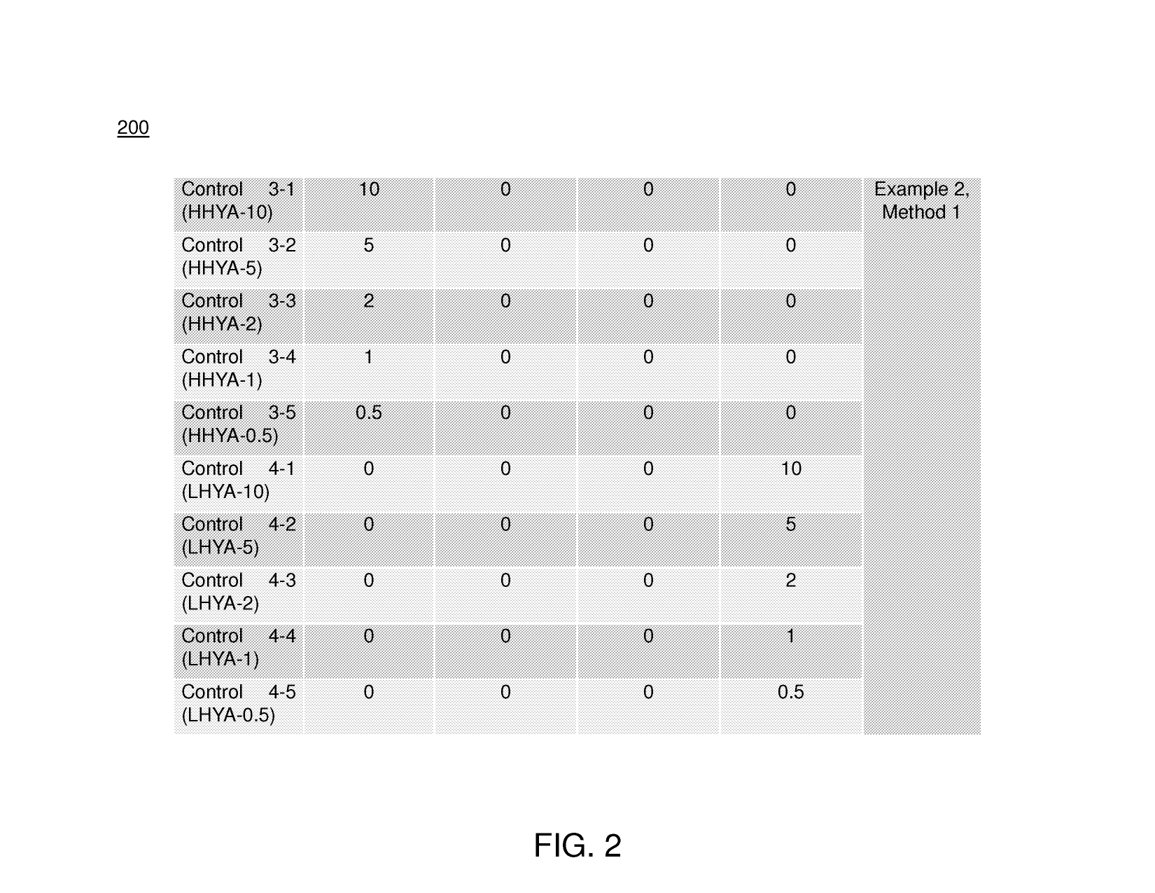

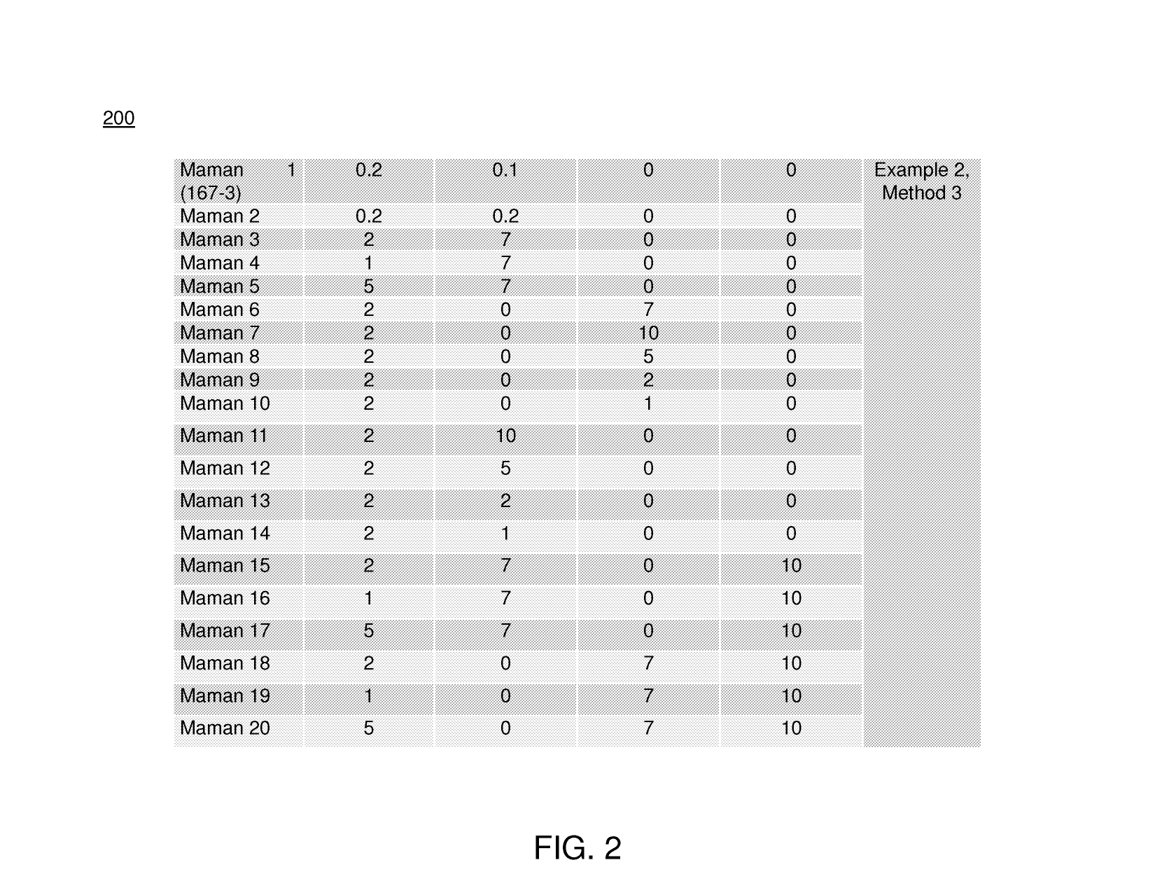

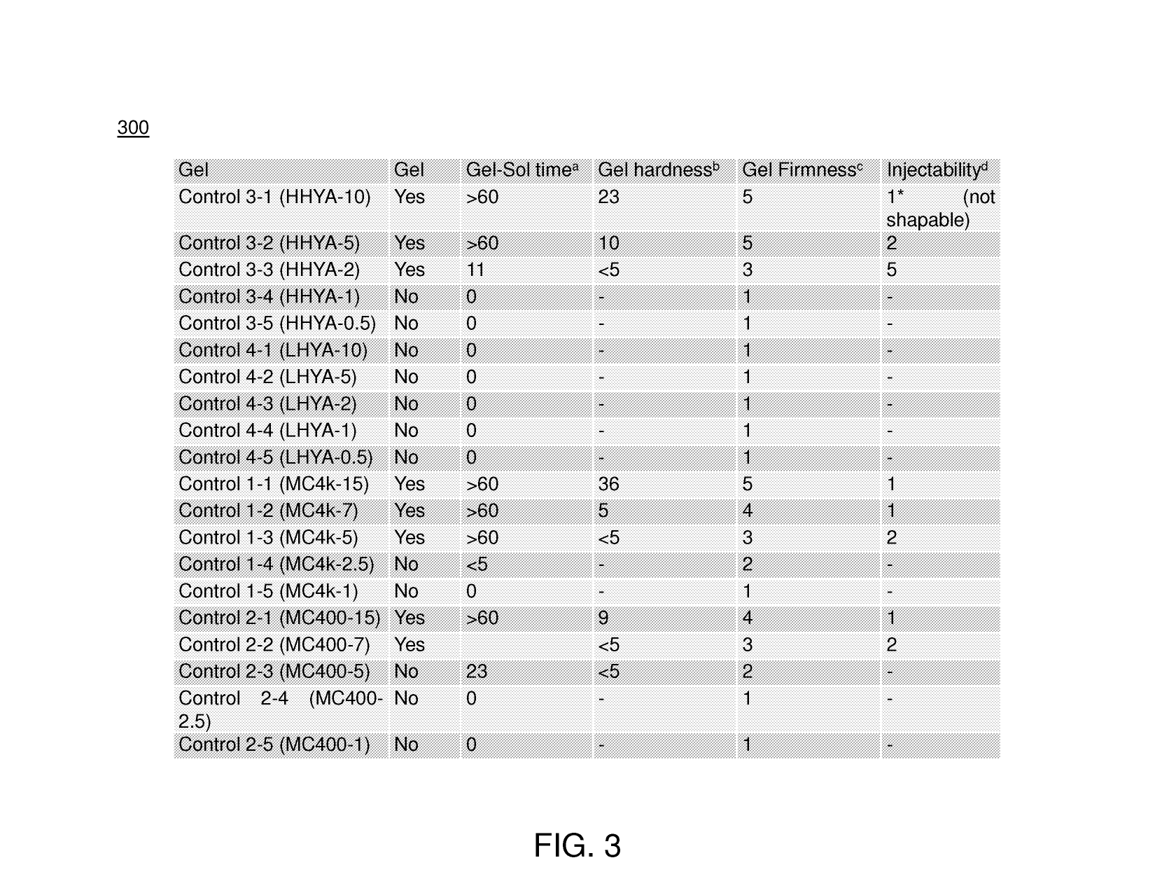

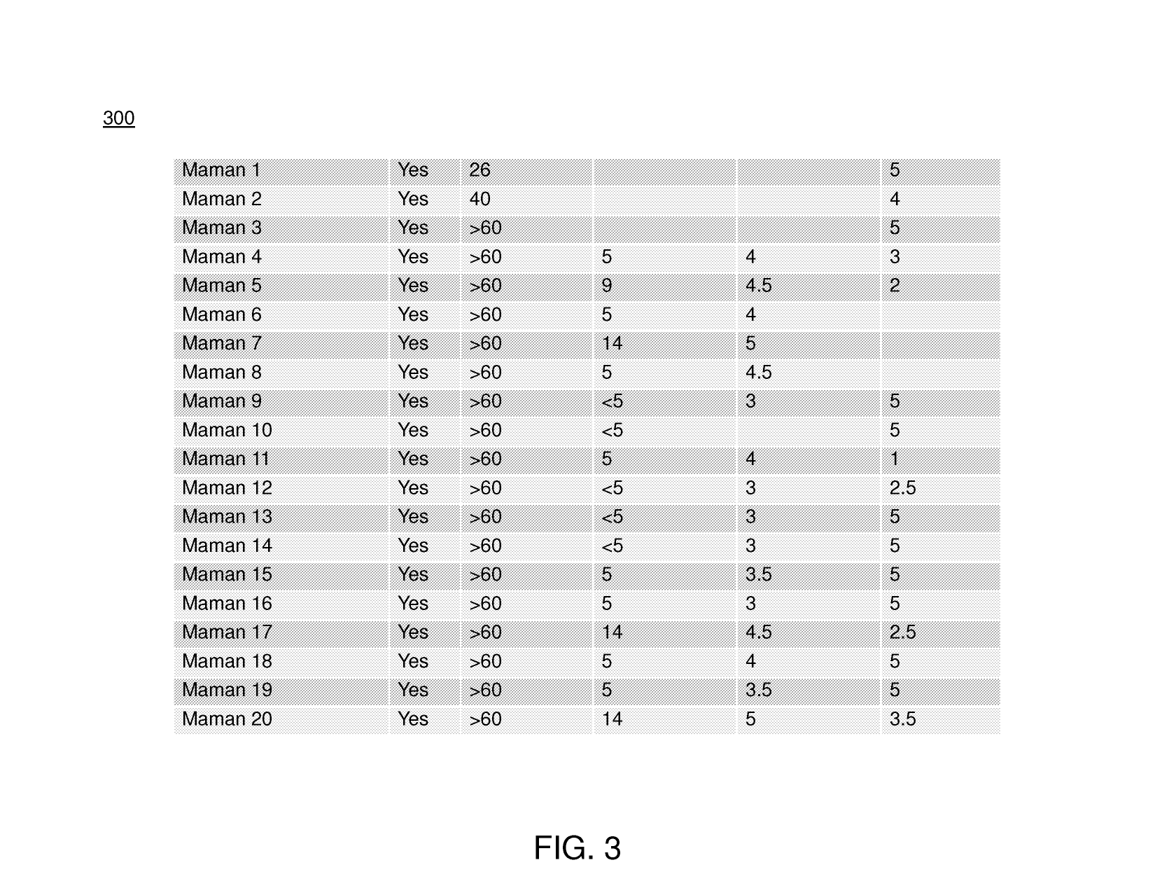

FIG. 2 illustrates a table 200 showing representative drug-free polymer composition matrices evaluated during formulation development, in accordance with embodiments of the present disclosure. The table 200 specifies weight percentages of the high-molecular-weight hyaluronic acid 106, the methylcellulose 108 of defined viscosity grades, and the low-molecular-weight hyaluronic acid 106, together with the preparation method employed. The table 200 defines the polymer concentration and ratio ranges investigated to assess their influence on resulting gel properties described with reference to FIG. 3.

FIG. 3 illustrates a table 300 showing experimental results demonstrating the influence of the injectable gel composition 102 on resulting gel properties, in accordance with various embodiment of the present disclosure. The table 300 indicates that the hyaluronic acid 106 or the methylcellulose 108 alone do not consistently produce gels simultaneously exhibiting prolonged gel–sol time, sufficient firmness, and acceptable injectability. In addition, the table 300 presents comparative measurements of injectability, gel–sol transition time, hardness, and firmness for selected formulations. The results illustrate that combinations of hyaluronic acid and methylcellulose yield gel properties that differ from those of either polymer alone, consistent with cooperative physical interactions between the polymers.

The table 300 illustrates representative sustained-release behavior of drug-loaded polymer matrices prepared in accordance with embodiments of the present disclosure, together with associated in vitro biological evaluation.

In certain embodiments, drug-loaded prototype matrices were prepared and evaluated for release characteristics under physiologically relevant conditions. Defined samples measuring approximately 0.5 cm² were cut from hydrated polymer sheets and suspended in approximately 1 mL of aqueous media selected from water, phosphate-buffered saline (PBS), or saline at approximately 37°C. Each formulation was evaluated in triplicate.

At pre-determined time intervals, aliquots of approximately 25–100 µL were withdrawn from the surrounding medium and analyzed using UV-visible spectroscopy to quantify drug concentration. Withdrawn volumes were replaced with fresh medium to maintain sink conditions. Sampling was continued until complete matrix dissolution or until the concentration of released drug fell below analytical detection limits. The time required for complete structural dissolution of the matrix was recorded.

The release profile shown in the table 300 reflects an initial release phase followed by a sustained release phase. In certain embodiments, an initial burst release occurs within approximately 1–30 minutes following introduction into aqueous media. Thereafter, sustained release proceeds over a period ranging from approximately 30 minutes to 14 days. The sustained phase is associated with progressive matrix hydration, dispersion, and, where applicable, degradation of encapsulation materials incorporated within the matrix.

Release kinetics are influenced by the quantity of drug loaded into the matrix and by the composition and physicochemical properties of any encapsulation technologies employed. The data demonstrate that the hydrated, physically assembled polymer network permits staged release behavior without reliance on irreversible crosslinked depots or long-term non-degrading implants.

In certain embodiments, representative formulations were evaluated for biological compatibility in vitro. Select ovarian and endometrial cell line cultures were incubated with prototype matrices under controlled laboratory conditions. Cell viability assays and hormone-related functional assays were performed at multiple time points.

The observed results indicated maintenance of cellular viability and functional responsiveness over the evaluated intervals. These findings are consistent with compatibility of the hydrated polymer matrix system with biologically active therapeutic agents and relevant tissue types.

Example 1 — Matrix Composition

Hyaluronic acid (HYA)–containing formulations were investigated as the basis of matrix materials for dissolving microneedles. Table 1 summarizes representative formulations prepared according to weight-percent (w/w%) ratios of dissolvable polymer components.

Example 2 — Preparation of Injectable Gels

Multiple preparation methods were evaluated for generating hydrated polymer gels.

### Method 1 — Hyaluronic Acid Gel Preparation

Hyaluronic acid gels were prepared by dissolving hyaluronic acid (mg) in approximately ten times the equivalent volume (mL) of hot water at about 65°C, followed by cooling to room temperature prior to use.

### Method 2 — Methylcellulose Gel Preparation

Methylcellulose gels were prepared by dispersing methylcellulose (mg) in approximately ten times the equivalent volume (mL) of hot water at about 65°C and allowing the dispersion to cool to room temperature with constant gentle shaking to promote uniform dissolution.

### Method 3 — Mixed Polymer Gel Preparation

Mixed gel formulations were prepared by combining dry powders of the two polymer components (mg), followed by addition of approximately ten times the equivalent volume (mL) of hot water at about 65°C and cooling to room temperature with constant gentle shaking.

The preparation methods generate hydrated polymer matrices formed through non-covalent physical interactions between polymer chains without chemical crosslinking.

### Example 4 — Preparation of Drug-Loaded Injectable Gel

Multiple approaches were evaluated for incorporating active pharmaceutical ingredients (APIs) into hydrated gel matrices.

### Method 1

Active pharmaceutical ingredients were directly stirred into gels previously prepared as described in Example 2.

### Method 2

Active pharmaceutical ingredients were encapsulated using methods known to those skilled in the art and gently mixed into gels prepared as described in Example 2.

### Method 3

Active pharmaceutical ingredients were introduced into gels using both direct incorporation and encapsulation approaches according to Methods 1 and 2.

Example 6 — Influence of Composition on Gel Properties

Gel properties were investigated as a function of injectability, gel time, and gel hardness. Results are presented in the table 200.

Gel–sol time was measured as the time required for 10 mL of gel to become flowable after inversion within a container of fixed dimensions.

Gel hardness was measured as the incremental weight required to flatten a 0.25 g sphere of gel into a 1 mm thick sheet using a standardized incremental loading method.

Gel firmness was evaluated by forming 0.25 g of gel into a sphere and scoring structural stability as follows:

5 = forms a sphere that holds its shape

4 = forms a sphere that is easily deformed

3 = does not hold a peak for more than 30 seconds

2 = threads with a peak

1 = liquid

Injectability was measured using a 1 mL sterile syringe fitted with a 30-gauge needle. The force required to expel gel using only thumb pressure while holding the syringe with one hand was scored as follows:

5 = flows easily through needle upon pushing

3 = flows through needle with increasing force

1 = does not flow regardless of force

Addition of an equivalent or lower weight percentage of low-viscosity methylcellulose (LMC; 41 kDa) relative to high-molecular-weight hyaluronic acid (HHYA) improved sol-gel behavior of HHYA gels while preserving injectability. However, these concentrations did not improve gel strength or firmness. Higher concentrations of LMC improved firmness but reduced injectability.

Replacement of LMC with higher-viscosity methylcellulose (HMC; 88 kDa) produced improved gel properties at lower methylcellulose concentrations, but again with reduced injectability.

Addition of low-molecular-weight hyaluronic acid (LHYA) to formulations resulted in improved flow properties despite increased solids content.

The data suggest that inclusion of the low-molecular-weight hyaluronic acid 106 modifies network organization in a manner that improves injectability despite increased total solids content.

Example 11 — Sustained Release Times of Drug-Loaded Gels

Prototype microneedle array patch samples measuring approximately 0.5 cm² were prepared and suspended in 1 mL of aqueous media selected from water, phosphate-buffered saline, and saline at approximately 37° C. Each sample was tested in triplicate.

At pre-determined time intervals, aliquots of approximately 25–100 µL were withdrawn and analyzed using UV-visible spectroscopy to quantify drug concentration in solution. Removed volumes were replaced with fresh medium. Sampling was repeated until complete dissolution of the patch matrices or until drug concentration fell below detection limits. Total dissolution time was recorded.

The microneedle array patches demonstrated burst release of incorporated drugs within approximately 1–30 minutes following exposure to aqueous media. Sustained release occurred thereafter as encapsulation materials degraded, providing release over approximately 30 minutes to 14 days. Release profiles varied depending on drug loading and encapsulation chemistry.

Example 12 — Safety and Efficacy of Formulations

Safety and efficacy of representative formulations were evaluated in vitro. Selected ovarian and endometrial cell line cultures were incubated with prototype microneedle array patches.

Cell viability and hormone production assays were performed at multiple time points.

The results demonstrated acceptable safety and efficacy profiles of the tested formulations over time.

The table 300 demonstrates that the hyaluronic acid 106 alone exhibits concentration-dependent gel formation but either lacks injectability at higher concentrations or fails to form stable gels at lower concentrations. The low molecular weight hyaluronic acid 106 alone does not form gels within the evaluated range. The methylcellulose 108 alone forms gels at sufficient concentrations but exhibits reduced injectability at higher viscosities and lower gel–sol stability at reduced concentrations. In contrast, the mixed formulations with the hyaluronic acid 106 and the methylcellulose 108 consistently form cohesive gels exhibiting prolonged gel–sol time (frequently exceeding 60 minutes), measurable hardness, and intermediate-to-high firmness and maintain improved injectability relative to single-polymer controls. Further, the data shows that inclusion of the low molecular weight hyaluronic acid 106 can improve injectability despite increased solids content. Collectively, the results establish that the gel performance is governed by cooperative physical interactions between the hyaluronic acid 106 and the methylcellulose 108 rather than by total polymer concentration alone. The table 300 evidences a non-linear structure–function relationship in which the defined polymer ratios and the molecular weight combinations produce balanced rheological properties suitable for injectable, short-term therapeutic delivery.

The injectable gel composition 102 provides a physically assembled, non-covalently structured hydrated matrix that enables controlled short-term delivery of large-molecule therapeutics without chemical crosslinking or irreversible depot formation. By combining high molecular weight hyaluronic acid with methylcellulose in defined ratios and viscosity ranges, the composition achieves the shear-responsive flow during administration and recovery of mechanical cohesion following injection. The decoupling of the injectability from residence time enables localized retention and maintain the ability to discontinue or adjust therapy without prolonged persistence. The present composition disperses, dilutes, or loses structural integrity within a defined window of approximately 1–14 days. The transient structural behavior supports therapeutic regimens requiring adjustable exposure, such as fertility-related protocols and other hormone-modulated treatments, and avoid long-term residual material accumulation in tissue. Furthermore, the matrix accommodates both directly dispersed and encapsulated therapeutic fractions within a single formulation. This enables staged release profiles with an early diffusion-mediated phase and a subsequent sustained phase governed by matrix hydration dynamics or short-term encapsulation degradation. The composition-of-matter solution integrates tunable rheology, controlled dissolution, and biologic compatibility within a single platform.

In embodiments of the present disclosure, the specific combination of non-crosslinked high molecular weight hyaluronic acid and methylcellulose produces measurable rheological and release-control effects not achievable by either polymer independently. First, the interaction between hydrated polymer chains generates a reversible physically assembled matrix characterized by shear-thinning flow behavior. Under applied shear stress during injection, temporary polymer chain alignment and partial disentanglement reduce apparent viscosity, enabling passage through fine-gauge needles. Upon cessation of shear, re-establishment of intermolecular interactions and chain entanglement restores mechanical cohesion at the administration site. This provides localized retention without chemical crosslinking.

Second, the matrix demonstrates controlled dispersion behavior governed by hydration-driven structural relaxation. The absence of covalent crosslinking permits predictable dilution and structural dissipation within a defined timeframe, thereby preventing long-term accumulation of polymer material.

Third, when therapeutic agents are incorporated in the dispersed and the encapsulated states, the matrix enables temporally separated release contributions. The directly dispersed fractions become available upon early hydration, and the encapsulated fractions are released upon short-term degradation or dissociation of carrier materials. The staged release mechanism supports maintenance of therapeutically effective concentrations and mitigate high initial peak exposure.

Collectively, the above mentioned effects arise from the defined polymer molecular weight ranges, viscosity grades, and concentration ratios, and provide a structurally grounded basis for the observed controlled, short-duration therapeutic delivery.

The injectable gel composition 102 may be utilized beyond fertility or hormone-related applications. In certain embodiments, the injectable gel composition 102 may serve as a delivery vehicle for other injectable biologics. The other injectable biologics may include peptides, monoclonal antibodies, growth factors, cytokines, or enzyme-based therapies. The transient residence profile and the tunable release behavior make the injectable gel composition 102 suitable for therapies requiring short-term localized exposure rather than prolonged depot action. In other embodiments, the injectable gel composition 102 may be adapted for tissue regeneration or wound-healing applications. The hydrophilic hyaluronic acid component 106 provides a hydrated microenvironment conducive to cell migration and matrix remodelling. The methylcellulose component 108 modulates structural stability during early healing phases. The matrix may serve as a temporary scaffold supporting localized delivery of regenerative signalling molecules before dispersing naturally. Also, the injectable gel composition 102 may be used in cosmetic or dermatological applications. The cosmetic or dermatological applications may include localized delivery of anti-aging peptides, dermal volumizing agents, anti-inflammatory biologics, or pigmentation-modulating compounds. The shear-thinning and reversible cohesion behavior allows minimally invasive administration and avoid long-term filler persistence associated with crosslinked hyaluronic acid products. Additionally, the injectable gel composition 102 may be used as a short-acting drug depot in contexts where adjustable therapy windows are required, including pain management, endocrine modulation beyond fertility indications, or localized anti-inflammatory treatment. The absence of irreversible crosslinking enables clinicians to modify dosing schedules without long-term polymer retention.

The present invention is described hereinafter by various embodiments. The invention may, however, be embodied in many different forms and should not be construed as limited to the embodiment set forth herein. Rather, the embodiment is provided so that this disclosure will be thorough and complete and will fully convey the scope of the invention to those skilled in the art. In the following detailed description, numeric values and ranges are provided for various aspects of the implementations described. These values and ranges are to be treated as examples only, and are not intended to limit the scope of the claims. In addition, a number of system architectures are identified as suitable for various facets of the implementations. These system architectures are to be treated as exemplary and are not intended to limit the scope of the invention.

The foregoing descriptions of specific embodiments of the present technology have been presented for purposes of illustration and description. They are not intended to be exhaustive or to limit the present technology to the precise forms disclosed, and obviously many modifications and variations are possible in light of the above teaching. The embodiments were chosen and described in order to best explain the principles of the present technology and its practical application, to thereby enable others skilled in the art to best utilize the present technology and various embodiments with various modifications as are suited to the particular use contemplated. It is understood that various omissions and substitutions of equivalents are contemplated as circumstance may suggest or render expedient, but such are intended to cover the application or implementation without departing from the spirit or scope of the claims of the present technology.

What is claimed is:

1. An injectable gel composition comprising:
(a) 0.5–20% w/w high molecular weight hyaluronic acid having a molecular weight of 1000–1400 kDa; and
(b) 0.5–20% w/w methylcellulose having a viscosity of 100–8000 cP,
wherein the hyaluronic acid is non-crosslinked,
wherein the hyaluronic acid and the methylcellulose form a physically assembled hydrated polymer matrix through reversible physical interactions and in an absence of added chemical crosslinking agents,
wherein the physically assembled hydrated polymer matrix is injectable,
wherein the injectable gel composition exhibits shear-responsive flow sufficient to permit injection through a needle or microneedle lumen and recovers mechanical cohesion upon cessation of shear, and
wherein the hydrated polymer matrix loses structural cohesion and disperses, dilutes, or degrades within 1–14 days following administration, without forming a chemically crosslinked long-acting polymer depot.

2. The injectable gel composition of claim 1, wherein the hyaluronic acid is present in an amount of 1–10% w/w.

3. The injectable gel composition of claim 1, wherein the methylcellulose is present in an amount of 1–10% w/w.

4. The injectable gel composition of claim 1, wherein the hyaluronic acid and methylcellulose are present in a weight ratio of 1:3 to 3:1.

5. The injectable gel composition of claim 1, further comprising 0.5–10% w/w low molecular weight hyaluronic acid having a molecular weight of 20–300 kDa.

6. The injectable gel composition of claim 1, wherein the methylcellulose has a viscosity of 400–4000 cP.

7. The injectable gel composition of claim 1, wherein the matrix loses structural cohesion within 1–7 days following administration.

8. The injectable gel composition of claim 1, further comprising a biodegradable polymer additive configured to degrade within 1–14 days following administration.

9. The injectable gel composition of claim 8, wherein the biodegradable polymer is selected from polyethylene glycol (PEG), poly(lactic-co-glycolic acid) (PLGA), polycaprolactone (PCL), or poly(lactic acid) (PLA).

10. The injectable gel composition of claim 1, further comprising a fatty acid or fatty-acid derivative configured to modulate transient microstructural organization within the matrix.

11. The injectable gel composition of claim 1, wherein the combined amount of high molecular weight hyaluronic acid and methylcellulose is 2–20% w/w of the injectable gel composition.

12. The injectable gel composition of claim 1, wherein the injectable gel composition is injectable through a needle having a gauge of 18–32.

13. The injectable gel composition of claim 1, wherein the hyaluronic acid and methylcellulose are combined with heated water at a temperature of 20–80°C during preparation of the injectable gel composition.

14. An injectable gel composition for controlled, short-term delivery of a fertility hormone to a subject, the injectable gel composition comprising:
(a) 0.5–20% w/w high molecular weight hyaluronic acid having a molecular weight of 1000–1400 kDa; and
(b) 0.5–20% w/w methylcellulose having a viscosity of 100–8000 cP,
wherein the hyaluronic acid is non-crosslinked,
wherein the hyaluronic acid and the methylcellulose form a physically assembled hydrated polymer matrix through reversible physical interactions and in an absence of added chemical crosslinking agents,
wherein the injectable gel composition exhibits shear-responsive flow sufficient to permit injection through a needle or microneedle lumen and recovers mechanical cohesion upon cessation of shear,
wherein the fertility hormone is present in unmodified form within the hydrated polymer matrix,
wherein the hydrated polymer matrix retains the fertility hormone at an administration site,
wherein the hydrated polymer matrix loses structural cohesion and disperses, dilutes, or loses structural integrity within 1–14 days following administration without formation of a covalently crosslinked polymer depot, and
wherein sustained hormone delivery is achieved over hours to days without irreversible commitment to prolonged exposure.

15. The injectable gel composition of claim 14, wherein the fertility hormone is dispersed directly within the hydrated matrix.

16. The injectable gel composition of claim 14, wherein at least a portion of the fertility hormone is encapsulated within biodegradable polymer particles dispersed within the matrix.

17. The injectable gel composition of claim 16, wherein the biodegradable polymer is selected from polyethylene glycol (PEG), poly(lactic-co-glycolic acid) (PLGA), polycaprolactone (PCL), or poly(lactic acid) (PLA).

18. The injectable gel composition of claim 14, wherein at least 75% of directly dispersed fertility hormone is released within 24 hours following administration.

19. The injectable gel composition of claim 14, wherein the fertility hormone is selected from follicle stimulating hormone (FSH), luteinizing hormone (LH), gonadotropin releasing hormone (GnRH), or progesterone.

20. The injectable gel composition of claim 14, wherein the injectable gel composition is configured to maintain therapeutically effective hormone levels for 1–7 days.
